(19) United States
(12) Patent Application Publication
(10) Pub. No.: US 2024/0392374 A1
(43) Pub. Date: Nov. 28, 2024

(54) DIAGNOSIS AND TREATMENT OF DISEASES AND CONDITIONS OF THE INTESTINAL TRACT

(71) Applicant: FLAGSHIP PIONEERING INNOVATIONS VI, LLC, Cambridge, MA (US)

(72) Inventors: Douglas Gowers COLE, Wayland, MA (US); Jason PARK, West Newton, MA (US); Sabrina Yusang YANG, Cambridge, MA (US); Boris HAYETE, Needham, MA (US); Andrew John GASPARRINI, Boston, MA (US)

(21) Appl. No.: 18/694,565

(22) PCT Filed: Sep. 23, 2022
(86) PCT No.: PCT/US2022/076927

§ 371 Date: Mar. 22, 2024

Related U.S. Application Data
(60) Provisional application No. 63/247,527, filed on Sep. 23, 2021.

(51) Int. Cl. C12Q 1/6883

(52) U.S. Cl. CPC C12Q 1/6883; C12Q 2600/118; C12Q 2600/158

(57) ABSTRACT

The invention relates to diagnostic and therapeutic methods for inflammatory bowel disease. Disclosed is a method of differentiating between ulcerative colitis (UC) and Crohn's disease (CD) in a subject having an inflammatory bowel disease (IBD) comprising determining a level of one or more of SEQ ID NOs: 1-590 in a sample from the subject.

### SEQUENCE LISTING

The instant application contains a Sequence Listing which has been submitted electronically in XML file format and is hereby incorporated by reference in its entirety. Said XML copy, created on Sep. 12, 2022, is named 51373-017WO1_Sequence_Listing_9_12_22.xml and is 8,441,923 bytes in size.

### FIELD OF THE INVENTION

The invention relates to diagnostic and therapeutic methods for inflammatory bowel disease.

### BACKGROUND

Mammals are colonized by microorganisms in the gastrointestinal (GI) tract, on the skin, and in other epithelial and tissue niches. The gastrointestinal tract of a healthy individual harbors an abundant and diverse microbial community. It is a complex system, providing an environment or niche for a community of many different species or organisms, including diverse strains of bacteria. Hundreds of different species may form a commensal community in the GI tract in a healthy person, and this complement of organisms evolves from the time of birth and is believed to form a functionally mature microbial population by about 3 years of age. Interactions between microbial strains in these populations and between microorganisms and the host, e.g., interactions with the host's immune system, shape the community structure, with availability of and competition for resources affecting the distribution of microorganisms.

A healthy microbiome may provide a subject with multiple benefits, including colonization resistance to a broad spectrum of pathogens, essential nutrient biosynthesis and absorption, and immune stimulation that plays a role in maintaining a healthy gut epithelium and appropriately controlled systemic immunity. Conversely, an unhealthy (e.g., dysregulated) microbiome may be associated with a disease state.

There is a need for methods for addressing problems in healthcare through assessment of the microbiome.

### SUMMARY OF THE INVENTION

In one aspect, the disclosure features a method of differentiating between a diagnosis of ulcerative colitis (UC) and Crohn's disease in a subject having an inflammatory bowel disease (IBD), the method comprising determining a level of one or more of SEQ ID NOs: 1-590 in a sample from the subject, wherein (a) a level of one or more of SEQ ID NOs: 1-590 that is changed relative to a respective reference level for SEQ ID NOs: 1-590 indicates that the subject is likely to have UC, and (b) a level of one or more of SEQ ID NOs: 1-590 that is not substantially changed relative to a respective reference level for SEQ ID NOs: 1-590 indicates that the subject is likely to have Crohn's disease.

A method for determining whether a subject having an IBD has UC or Crohn's disease, comprising the steps of (a) measuring the nucleic acid level of one or more of SEQ ID NOs: 1-590 in a sample collected from the subject using amplification and one or more pairs of primers specific for each of the one or more of SEQ ID NOs: 1-590; and (b) using the amplification results to determine whether the level of one or more of SEQ ID NOs: 1-590 is changed relative to a respective reference level for SEQ ID NOs: 1-590, wherein (i) a level of one or more of SEQ ID NOs: 1-590 that is changed relative to a respective reference level for SEQ ID NOs: 1-590 indicates that the subject is likely to have UC, and (ii) a level of one or more of SEQ ID NOs: 1-590 that is not substantially changed relative to a respective reference level for SEQ ID NOs: 1-590 indicates that the subject is likely to have Crohn's disease, thereby determining whether the subject has UC or Crohn's disease.

In some embodiments, the method comprises determining or measuring a level of at least two, at least three, at least four, at least five, at least six, at least seven, at least eight, at least nine, at least ten, at least fifteen, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, or at least 585 of SEQ ID NOs: 1-590 in the sample from the subject.

In some embodiments, the method comprises determining or measuring a level of all 590 of SEQ ID NOs: 1-590 in the sample from the subject.

In some embodiments, a level of one or more of SEQ ID NOs: 1-590 is changed relative to the respective reference level for SEQ ID NO: 1-590 in the sample from the subject and the method further comprises administering a therapy appropriate for treating UC to the subject.

In some embodiments, a level of one or more of SEQ ID NOs: 1-590 is not substantially changed relative to the respective reference level for SEQ ID NO: 1-590 in the sample from the subject and the method further comprises administering a therapy appropriate for treating Crohn's disease to the subject.

In some embodiments, the method further comprises treatment by fecal microbiota transplant (FMT).

In some embodiments, the reference level is a pre-assigned level.

In some embodiments, the reference level is a level in a set of samples from a reference population. In some embodiments, the reference population is a population of subjects having Crohn's disease.

In some embodiments, the level of each of SEQ ID NOs: 1-590 that is determined or measured in the sample from the subject is a nucleic acid level. In some embodiments, the nucleic acid level is a DNA level.

In some embodiments, the change is a decrease relative to the reference level. In some embodiments, the levels of at least 10%, at least 20%, at least 30%, at least 40%, at least 50%, at least 60%, at least 70%, at least 80%, at least 90%, or more than 90% of the sequences for which a level is determined or measured are decreased relative to a respective reference level for the sequence.

In some embodiments, the change is an increase relative to the reference level. In some embodiments, the levels of at least 10%, at least 20%, at least 30%, at least 40%, at least 50%, at least 60%, at least 70%, at least 80%, at least 90%, or more than 90% of the sequences for which a level is determined or measured are increased relative to a respective reference level for the sequence.

In some embodiments, the sample comprises a sample of the microbiota of the subject.

In some embodiments, the sample is a fecal sample.

In another aspect, the disclosure features a kit for differentiating between a diagnosis of ulcerative colitis (UC) and Crohn's disease in a subject having an inflammatory bowel disease (IBD), the kit comprising: (a) polypeptides or polynucleotides capable of determining the level of one or more of SEQ ID NOs: 1-590 in a sample from the subject; and (b) instructions for use of the polypeptides or polynucleotides to determine the level of one or more of SEQ ID NOs: 1-590 in the sample from the subject, wherein (a) a level of one or more of SEQ ID NOs: 1-590 that is changed relative to a respective reference level for SEQ ID NOs: 1-590 indicates that the subject is likely to have UC, and (b) a level of one or more of SEQ ID NOs: 1-590 that is not substantially changed relative to a respective reference level for SEQ ID NOs: 1-590 indicates that the subject is likely to have Crohn's disease.

In another aspect, the disclosure features a method of differentiating between a diagnosis of ulcerative colitis (UC) and Crohn's disease in a subject having an inflammatory bowel disease (IBD), the method comprising determining a level of each of SEQ ID NOs: 1-590 in a sample from the subject, wherein (a) a level of at least 50% of SEQ ID NOs: 1-590 that is changed relative to a respective reference level for SEQ ID NOs: 1-590 indicates that the subject is likely to have UC, and (b) a level of a level of at least 50% of SEQ ID NOs: 1-590 that is not substantially changed relative to a respective reference level for SEQ ID NOs: 1-590 indicates that the subject is likely to have Crohn's disease.

In some embodiments, a level of at least 50% of SEQ ID NOs: 1-590 is changed relative to a respective reference level for SEQ ID NOs: 1-590 in the sample from the subject and the method further comprises administering an anti-UC therapy to a subject.

In some embodiments, a level of at least 50% of SEQ ID NOs: 1-590 is not substantially changed relative to a respective reference level for SEQ ID NOs: 1-590 in the sample from the subject and the method further comprises administering an anti-Crohn's disease therapy to a subject. In another aspect, the disclosure features a method of treating a subject having UC using a therapy appropriate for treating UC, wherein the subject was diagnosed as having UC by any of the methods provided herein.

In another aspect, the disclosure features a method of treating a subject having Crohn's disease using a therapy appropriate for treating Crohn's disease, wherein the subject was diagnosed as having Crohn's disease by any of the methods provided herein.

In another aspect, the disclosure features a method of treating a subject having UC using a therapy appropriate for treating UC, the method comprising the steps of (a) measuring the nucleic acid level of one or more of SEQ ID NOs: 1-590 in a sample collected from the subject using amplification and one or more pairs of primers specific for each of the one or more of SEQ ID NOs: 1-590; (b) using the amplification results to determine whether the level of one or more of SEQ ID NOs: 1-590 is changed relative to a respective reference level for SEQ ID NOs: 1-590, wherein a level of one or more of SEQ ID NOs: 1-590 that is changed relative to a respective reference level for SEQ ID NOs: 1-590 indicates that the subject is likely to have UC; and (c) treating a subject who has been determined to be likely to have UC with a therapy appropriate for treating UC.

In another aspect, the disclosure features a method of treating a subject having Crohn's disease using a therapy appropriate for treating Crohn's disease, the method comprising the steps of (a) measuring the nucleic acid level of one or more of SEQ ID NOs: 1-590 in a sample collected from the subject using amplification and one or more pairs of primers specific for each of the one or more of SEQ ID NOs: 1-590; (b) using the amplification results to determine whether the level of one or more of SEQ ID NOs: 1-590 is changed relative to a respective reference level for SEQ ID NOs: 1-590, wherein a level of one or more of SEQ ID NOs: 1-590 that is not substantially changed relative to a respective reference level for SEQ ID NOs: 1-590 indicates that the subject is likely to have Crohn's disease; and (c) treating a subject who has been determined to be likely to have Crohn's disease with a therapy appropriate for treating Crohn's disease.

In some embodiments, the nucleic acid level of the one or more of SEQ ID NOs: 1-590 in the sample collected from the subject are measured using amplification and one or more pairs of primers specific for each of the one or more of SEQ ID NOs: 1-590, and the amplification results are used to determine whether the level of one or more of SEQ ID NOs: 1-590 is changed relative to a respective reference level for SEQ ID NOs: 1-590.

In some embodiments, the method comprises measuring the nucleic acid level of at least two, at least three, at least four, at least five, at least six, at least seven, at least eight, at least nine, at least ten, at least fifteen, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, or at least 585 of SEQ ID NOs: 1-590 in the sample from the subject. In some embodiments, the method comprises measuring the nucleic acid level of all 590 of SEQ ID NOs: 1-590 in the sample from the subject.

In some embodiments, the reference level is a pre-assigned level. In some embodiments, the reference level is a level in a set of samples from a reference population. In some embodiments, the reference population is a population of healthy subjects.

In some embodiments, the nucleic acid level is a DNA level.

In some embodiments, the change is a decrease relative to the reference level. In some embodiments, the levels of at least 10%, at least 20%, at least 30%, at least 40%, at least 50%, at least 60%, at least 70%, at least 80%, at least 90%, or more than 90% of the sequences for which a level is measured are decreased relative to a respective reference level for the sequence.

In some embodiments, the change is an increase relative to the reference level. In some embodiments, the levels of at least 10%, at least 20%, at least 30%, at least 40%, at least 50%, at least 60%, at least 70%, at least 80%, at least 90%, or more than 90% of the sequences for which a level is measured are increased relative to a respective reference level for the sequence.

In some embodiments, the sample comprises a sample of the microbiota of the subject. In some embodiments, the sample is a fecal sample.

In another aspect, the disclosure features a method of treating a subject having UC, the method comprising the steps of (a) measuring the nucleic acid level of each of SEQ ID NOs: 1-590 in a sample collected from the subject, wherein a level of at least 50% of SEQ ID NOs: 1-590 that is changed relative to a respective reference level for SEQ ID NOs: 1-590 indicates that the subject is likely to have UC, thereby determining an increased risk of UC for the subject; and (b) treating a subject who has been determined to have an increased risk of UC with a therapy appropriate for treating UC.

In another aspect, the disclosure features a method of treating a subject having Crohn's disease, the method comprising the steps of (a) measuring the nucleic acid level of each of SEQ ID NOs: 1-590 in a sample collected from the subject, wherein a level of at least 50% of SEQ ID NOs: 1-590 that is not substantially changed relative to a respective reference level for SEQ ID NOs: 1-590 indicates that the subject is likely to have Crohn's disease, thereby determining an increased risk of Crohn's disease for the subject; and (b) treating a subject who has been determined to have an increased risk of Crohn's disease with a therapy appropriate for treating Crohn's disease.

In some embodiments, the nucleic acid levels of each of SEQ ID NOs: 1-590 in the sample collected from the subject are measured using amplification and one or more pairs of primers specific for each of SEQ ID NOs: 1-590, and the amplification results are used to determine whether the level of each of SEQ ID NOs: 1-590 is changed relative to a respective reference level for SEQ ID NOs: 1-590.

### BRIEF DESCRIPTION OF THE DRAWINGS

FIG. 1 is a graph showing the out-of-cohort performance (as percent specificity and sensitivity) of metagenomic markers for distinguishing individual having ulcerative colitis (UC) from individuals having Crohn's disease. AUC=area under curve.

FIG. 2 is a heat map showing the level (scaled reads per kilobase of transcript per million mapped reads (RPKM)) of all significant metagenomic markers in patients having UC or Crohn's disease (CD).

FIG. 3 is a heat map showing the level (scaled reads per kilobase of transcript per million mapped reads (RPKM)) of all significant metagenomic markers in patients having UC or CD.

### DETAILED DESCRIPTION OF THE INVENTION

### Definitions

The term “changed,” as used herein, refers to an observable difference in the level of a marker in a subject (e.g., in a sample from the subject), as determined using techniques and methods known in the art for the measurement of the marker. A marker level that is changed in a subject may result in a difference of at least 1% (e.g., at least 5%, 10%, 15%, 20%, 25%, 30%, 35%, 40%, 45%, 50%, 55%, 60%, 65%, 70%, 75%, 80%, 85%, 90%, 95%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) more or less than a reference level (e.g., a level from a healthy subject or a level prior to treatment) (e.g., up to 100% or up to 100-fold relative to the reference level). In some embodiments, the change is an increase in the level of a marker in a subject. Increasing the marker level in a subject may result in an increase of at least 1% (e.g., at least 5%, 10%, 15%, 20%, 25%, 30%, 35%, 40%, 45%, 50%, 55%, 60%, 65%, 70%, 75%, 80%, 85%, 90%, 95%, 98%, 99%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the reference level (e.g., up to 100% or up to 100-fold relative to the reference level). In other embodiments, the change is a decrease the level of a marker in a subject. Decreasing the marker level in a subject may result in a decrease of at least 1% (e.g., at least 5%, 10%, 15%, 20%, 25%, 30%, 35%, 40%, 45%, 50%, 55%, 60%, 65%, 70%, 75%, 80%, 85%, 90%, 95%, or 100%, or at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the reference level (e.g., up to 100% or up to 100-fold relative to the reference level).

In some embodiments, the change in the level of a portion of the markers analyzed is an increase, while the change in the level of another portion of the markers analyzed is a decrease. In some embodiments, the change in at least 10%, 20%, 30%, 40%, 50%, 60%, 70%, 80%, 90%, 95%, or more (e.g., 100%) of the markers analyzed is an increase relative to a reference level. In some embodiments, the change in at least 10%, 20%, 30%, 40%, 50%, 60%, 70%, 80%, 90%, 95%, or more (e.g., 100%) of the markers analyzed is a decrease relative to a reference level.

The term “pharmaceutical composition.” as used herein, represents a composition formulated with a pharmaceutically acceptable excipient. For example, a “pharmaceutical composition” can be a composition that is manufactured or sold with the approval of a governmental regulatory agency as part of a therapeutic regimen for the treatment of a disease, disorder, or condition in a mammal, intended for such use, or in development for such use. In some examples, the pharmaceutical composition is a pre-approved composition.

The term “subject,” as used herein, represents a human or non-human animal (e.g., a mammal).

“Treatment” and “treating,” as used herein, refer to the medical management of a subject with the intent to improve, ameliorate, stabilize, prevent, or cure a disease, disorder, or condition. This term includes active treatment (treatment directed to improve the disease, disorder, or condition); causal treatment (treatment directed to the cause of the associated disease, disorder, or condition); palliative treatment (treatment designed for the relief of symptoms of the disease, disorder, or condition); preventative treatment (treatment directed to minimizing or partially or completely inhibiting the development of the associated disease, disorder, or condition); and supportive treatment (treatment employed to supplement another therapy).

The term “inflammatory bowel disease” or “IBD,” as used herein, refers to a condition of the bowel, e.g., the small intestine, large intestine, mouth, esophagus, stomach, rectum, and/or anus, that is characterized by inflammation. Examples of IBD include ulcerative colitis (UC) and Crohn's disease. Other examples include microscopic colitis (e.g., collagenous colitis or lyphocytic colitis), diversion colitis, Behçet's disease, or indeterminate colitis.

### I. METHODS FOR DIFFERENIATING BETWEEN DIAGNOSES OF ULCERATIVE COLITIS AND CROHN'S DISEASE

The invention is based, in part, on the discovery that levels of gut microbiome biomarkers (i.e., markers of bacterial origin), which may be referred to as co-evolved molecules, can be used to distinguish between patients having ulcerative colitis (UC) and Crohn's disease. Accordingly, the disclosure provides methods of diagnosing, treating, and monitoring subjects (e.g., human patients) based on this discovery.

### Diagnostic Methods and Methods of Treatment

In some aspects, the disclosure features methods of differentiating between a diagnosis of ulcerative colitis (UC) and Crohn's disease in a subject having an inflammatory bowel disease (IBD), the method comprising determining a level of one or more of SEQ ID NOs: 1-590 in a sample from the subject, wherein (a) a level of one or more of SEQ ID NOs: 1-590 that is changed relative to a respective reference level for SEQ ID NOs: 1-590 indicates that the subject is likely to have UC, and (b) a level of one or more of SEQ ID NOs: 1-590 that is not substantially changed relative to a respective reference level for SEQ ID NOs: 1-590 indicates that the subject is likely to have Crohn's disease. SEQ ID NOs: 1-590 are bacterial sequences.

In some aspects, the disclosure features a method for determining whether a subject having an IBD has UC or Crohn's disease, comprising the steps of: (a) measuring the nucleic acid level of one or more of SEQ ID NOs: 1-590 in a sample collected from the subject using amplification and one or more pairs of primers specific for each of the one or more of SEQ ID NOs: 1-590; and (b) using the amplification results to determine whether the level of one or more of SEQ ID NOs: 1-590 is changed relative to a respective reference level for SEQ ID NOs: 1-590, wherein (i) a level of one or more of SEQ ID NOs: 1-590 that is changed relative to a respective reference level for SEQ ID NOs: 1-590 indicates that the subject is likely to have UC, and (ii) a level of one or more of SEQ ID NOs: 1-590 that is not substantially changed relative to a respective reference level for SEQ ID NOs: 1-590 indicates that the subject is likely to have Crohn's disease, thereby determining whether the subject has UC or Crohn's disease.

In some aspects, the disclosure features a method of treating a subject having UC using a therapy appropriate for treating UC, wherein the subject was diagnosed as having UC by any of the methods provided herein.

In some aspects, the disclosure features a method of treating a subject having Crohn's disease using a therapy appropriate for treating Crohn's disease, wherein the subject was diagnosed as having Crohn's disease by any of the methods provided herein.

In some aspects, the disclosure features a method of treating a subject having UC using a therapy appropriate for treating UC, the method comprising the steps of (a) measuring the nucleic acid level of one or more of SEQ ID NOs: 1-590 in a sample collected from the subject using amplification and one or more pairs of primers specific for each of the one or more of SEQ ID NOs: 1-590; (b) using the amplification results to determine whether the level of one or more of SEQ ID NOs: 1-590 is changed relative to a respective reference level for SEQ ID NOs: 1-590, wherein a level of one or more of SEQ ID NOs: 1-590 that is changed relative to a respective reference level for SEQ ID NOs: 1-590 indicates that the subject is likely to have UC; and (c) treating a subject who has been determined to be likely to have UC with a therapy appropriate for treating UC.

In some aspects, the disclosure features a method of treating a subject having Crohn's disease using a therapy appropriate for treating Crohn's disease, the method comprising the steps of (a) measuring the nucleic acid level of one or more of SEQ ID NOs: 1-590 in a sample collected from the subject using amplification and one or more pairs of primers specific for each of the one or more of SEQ ID NOs: 1-590; (b) using the amplification results to determine whether the level of one or more of SEQ ID NOs: 1-590 is changed relative to a respective reference level for SEQ ID NOs: 1-590, wherein a level of one or more of SEQ ID NOs: 1-590 that is not substantially changed relative to a respective reference level for SEQ ID NOs: 1-590 indicates that the subject is likely to have Crohn's disease; and (c) treating a subject who has been determined to be likely to have Crohn's disease with a therapy appropriate for treating Crohn's disease.

In some aspects, the disclosure features a method of treating a subject having UC, the method comprising the steps of (a) measuring the nucleic acid level of each of SEQ ID NOs: 1-590 in a sample collected from the subject, wherein a level of at least 50% of SEQ ID NOs: 1-590 that is changed relative to a respective reference level for SEQ ID NOs: 1-590 indicates that the subject is likely to have UC, thereby determining an increased risk of UC for the subject; and (b) treating a subject who has been determined to have an increased risk of UC with a therapy appropriate for treating UC.

In some aspects, the disclosure features a method of treating a subject having Crohn's disease, the method comprising the steps of (a) measuring the nucleic acid level of each of SEQ ID NOs: 1-590 in a sample collected from the subject, wherein a level of at least 50% of SEQ ID NOs: 1-590 that is not substantially changed relative to a respective reference level for SEQ ID NOs: 1-590 indicates that the subject is likely to have Crohn's disease, thereby determining an increased risk of Crohn's disease for the subject; and (b) treating a subject who has been determined to have an increased risk of Crohn's disease with a therapy appropriate for treating Crohn's disease.

In some embodiments, the sample comprises a sample of the microbiota (e.g., the gut microbiota) of the subject. In some embodiments, the sample is a fecal sample. In some embodiments, the sample is a colon or rectal biopsy.

In some embodiments, the method comprises determining or measuring a level of at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, at least 500, at least 505, at least 510, at least 515, at least 520, at least 525, at least 530, at least 535, at least 540, at least 545, at least 550, at least 555, at least 560, at least 565, at least 570, at least 575, at least 580, or at least 585 of SEQ ID NOs: 1-590 in a sample from the subject, e.g., comprises determining or measuring a level of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 215-220, 220-225, 225-230, 230-235, 235-240, 240-245, 245-250, 250-255, 255-260, 260-265, 265-270, 270-275, 275-280, 280-285, 285-290, 290-295, 295-300, 300-305, 305-310, 310-315, 315-320, 320-325, 325-330, 330-335, 335-340, 340-345, 345-350, 350-355, 355-360, 360-365, 365-370, 370-375, 375-380, 380-385, 385-390, 390-395, 395-400, 400-405, 405-410, 410-415, 415-420, 420-425, 425-430, 430-435, 435-440, 440-445, 445-450, 450-455, 455-460, 460-465, 465-470, 470-475, 475-480, 480-485, 485-490, 490-495, 495-500, 500-505, 505-510, 510-515, 515-520, 520-525, 525-530, 530-535, 535-540, 540-545, 545-550, 550-555, 560-565, 565-570, 570-575, 575-580, 580-585, or 585-590 of SEQ ID NOs: 1-590 in the sample from the subject. In some embodiments, the method comprises determining or measuring a level of all 590 of SEQ ID NOs: 1-590 in a sample from the subject.

In some embodiments, a change in the level of at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, at least 500, at least 505, at least 510, at least 515, at least 520, at least 525, at least 530, at least 535, at least 540, at least 545, at least 550, at least 555, at least 560, at least 565, at least 570, at least 575, at least 580, or at least 585 of SEQ ID NOs: 1-590 in a sample from the subject, e.g., a change in the level of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 215-220, 220-225, 225-230, 230-235, 235-240, 240-245, 245-250, 250-255, 255-260, 260-265, 265-270, 270-275, 275-280, 280-285, 285-290, 290-295, 295-300, 300-305, 305-310, 310-315, 315-320, 320-325, 325-330, 330-335, 335-340, 340-345, 345-350, 350-355, 355-360, 360-365, 365-370, 370-375, 375-380, 380-385, 385-390, 390-395, 395-400, 400-405, 405-410, 410-415, 415-420, 420-425, 425-430, 430-435, 435-440, 440-445, 445-450, 450-455, 455-460, 460-465, 465-470, 470-475, 475-480, 480-485, 485-490, 490-495, 495-500, 500-505, 505-510, 510-515, 515-520, 520-525, 525-530, 530-535, 535-540, 540-545, 545-550, 550-555, 560-565, 565-570, 570-575, 575-580, 580-585, or 585-590 of SEQ ID NOs: 1-590 in the sample from the subject relative to a respective reference level for SEQ ID NOs: 1-590 indicates that the subject is likely to have UC. In some embodiments, a change in the level of all 590 of SEQ ID NOs: 1-590 relative to a respective reference level for SEQ ID NOs: 1-590 indicates that the subject is likely to have UC.

In some embodiments, a change in the level of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, at least 98%, at least 99%, or 100% of the total number of SEQ ID NOs: 1-590 measured in a sample from the subject relative to a respective reference level for SEQ ID NOs: 1-590 indicates that the subject is likely to have UC.

In some embodiments, a threshold number of one or more of SEQ ID NOs: 1-590 is changed relative to the respective reference level for SEQ ID NO: 1-590 in the sample from the subject (e.g., a number of SEQ ID NOs: 1-590 that has been determined to indicate that the patient is likely to have UC, e.g., a level of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, at least 500, at least 505, at least 510, at least 515, at least 520, at least 525, at least 530, at least 535, at least 540, at least 545, at least 550, at least 555, at least 560, at least 565, at least 570, at least 575, at least 580, or at least 585 of SEQ ID NOs: 1-590 in a sample from the subject (e.g., a level of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 215-220, 220-225, 225-230, 230-235, 235-240, 240-245, 245-250, 250-255, 255-260, 260-265, 265-270, 270-275, 275-280, 280-285, 285-290, 290-295, 295-300, 300-305, 305-310, 310-315, 315-320, 320-325, 325-330, 330-335, 335-340, 340-345, 345-350, 350-355, 355-360, 360-365, 365-370, 370-375, 375-380, 380-385, 385-390, 390-395, 395-400, 400-405, 405-410, 410-415, 415-420, 420-425, 425-430, 430-435, 435-440, 440-445, 445-450, 450-455, 455-460, 460-465, 465-470, 470-475, 475-480, 480-485, 485-490, 490-495, 495-500, 500-505, 505-510, 510-515, 515-520, 520-525, 525-530, 530-535, 535-540, 540-545, 545-550, 550-555, 560-565, 565-570, 570-575, 575-580, 580-585, or 585-590)) of SEQ ID NOs: 1-590 is changed relative to the respective reference level for SEQ ID NO: 1-590 in the sample from the subject and the method further comprises administering a therapy appropriate for treating UC to a subject.

The anti-UC therapy may be any medicament, treatment, or combination thereof suitable for the treatment of UC. For example, the therapy may include an anti-inflammatory drug (e.g., a corticosteroid or oral 5-aminosalicylate (e.g., sulfasalazine or mesalamine)), an immune system suppressor (e.g., azathioprine, mercaptopurine, or methotrexate), a steroid, an analgesic, a biologic (e.g., natalizumab, vedolizumab, infliximab, adalimumab, certolizumab pegol, or ustekinumab), an antibiotic (e.g., ciprofloxacin or metronidazole), and/or surgery (also see the list provided below). Any one or more of these therapies may optionally be used in any of the methods described herein as employing a therapy that is appropriate for treating UC.

In some aspects, the anti-UC therapy further comprises treatment by fecal microbiota transplant (FMT).

In some embodiments, a level of at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, at least 500, at least 505, at least 510, at least 515, at least 520, at least 525, at least 530, at least 535, at least 540, at least 545, at least 550, at least 555, at least 560, at least 565, at least 570, at least 575, at least 580, or at least 585 of SEQ ID NOs: 1-590 in a sample from the subject, e.g., a change in the level of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 215-220, 220-225, 225-230, 230-235, 235-240, 240-245, 245-250, 250-255, 255-260, 260-265, 265-270, 270-275, 275-280, 280-285, 285-290, 290-295, 295-300, 300-305, 305-310, 310-315, 315-320, 320-325, 325-330, 330-335, 335-340, 340-345, 345-350, 350-355, 355-360, 360-365, 365-370, 370-375, 375-380, 380-385, 385-390, 390-395, 395-400, 400-405, 405-410, 410-415, 415-420, 420-425, 425-430, 430-435, 435-440, 440-445, 445-450, 450-455, 455-460, 460-465, 465-470, 470-475, 475-480, 480-485, 485-490, 490-495, 495-500, 500-505, 505-510, 510-515, 515-520, 520-525, 525-530, 530-535, 535-540, 540-545, 545-550, 550-555, 560-565, 565-570, 570-575, 575-580, 580-585, or 585-590 of SEQ ID NOs: 1-590 in the sample from the subject that is not substantially changed relative to a respective reference level for SEQ ID NOs: 1-590 indicates that the subject is likely to have Crohn's disease. In some embodiments, a level of all 590 of SEQ ID NOs: 1-590 that is not substantially changed relative to a respective reference level for SEQ ID NOs: 1-590 indicates that the subject is likely to have Crohn's disease.

In some embodiments, a level of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 95%, at least 98%, at least 99%, or 100% of the total number of SEQ ID NOs: 1-590 measured in a sample from the subject that is not substantially changed relative to a respective reference level for SEQ ID NOs: 1-590 indicates that the subject is likely to have Crohn's disease.

In some embodiments, a threshold number of one or more of SEQ ID NOs: 1-590 is not substantially changed relative to the respective reference level for SEQ ID NO: 1-590 in the sample from the subject (e.g., a number of SEQ ID NOs: 1-590 that has been determined to indicate that the patient is likely to have Crohn's disease, e.g., a level of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, at least 400, at least 405, at least 410, at least 415, at least 420, at least 425, at least 430, at least 435, at least 440, at least 445, at least 450, at least 455, at least 460, at least 465, at least 470, at least 475, at least 480, at least 485, at least 490, at least 495, at least 500, at least 505, at least 510, at least 515, at least 520, at least 525, at least 530, at least 535, at least 540, at least 545, at least 550, at least 555, at least 560, at least 565, at least 570, at least 575, at least 580, or at least 585 of SEQ ID NOs: 1-590 in a sample from the subject (e.g., a level of 1-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-215, 215-220, 220-225, 225-230, 230-235, 235-240, 240-245, 245-250, 250-255, 255-260, 260-265, 265-270, 270-275, 275-280, 280-285, 285-290, 290-295, 295-300, 300-305, 305-310, 310-315, 315-320, 320-325, 325-330, 330-335, 335-340, 340-345, 345-350, 350-355, 355-360, 360-365, 365-370, 370-375, 375-380, 380-385, 385-390, 390-395, 395-400, 400-405, 405-410, 410-415, 415-420, 420-425, 425-430, 430-435, 435-440, 440-445, 445-450, 450-455, 455-460, 460-465, 465-470, 470-475, 475-480, 480-485, 485-490, 490-495, 495-500, 500-505, 505-510, 510-515, 515-520, 520-525, 525-530, 530-535, 535-540, 540-545, 545-550, 550-555, 560-565, 565-570, 570-575, 575-580, 580-585, or 585-590)) of SEQ ID NOs: 1-590 is not substantially changed relative to the respective reference level for SEQ ID NO: 1-590 in the sample from the subject and the method further comprises administering a therapy appropriate for treating Crohn's disease to a subject.

The anti-Crohn's disease therapy may be any medicament, treatment, or combination thereof suitable for the treatment of Crohn's disease. For example, the therapy may include an anti-inflammatory drug (e.g., a nonsteroidal anti-inflammatory drug), an immune system suppressor, a steroid, an analgesic, an antibiotic, and/or surgery. In some aspects, the anti-Crohn's disease therapy further comprises treatment by fecal microbiota transplant (FMT). Any one or more of these types of therapies may optionally be used in any of the methods described herein as employing a therapy appropriate for treating Crohn's disease.

Further agents that may be used for the treatment of an IBD (e.g., UC or Crohn's disease) include a biologic therapy (e.g., vedolizumab (ENTYVIO®), infliximab (REMICADE®), adalimumab (HUMIRA®), golimumab (SIMPONI®), certolizumab (CIMZIA®), risankizumab (SKYRIZI®), or ustekinumab (STELARA®)); an anti-inflammatory agent (e.g., a corticosteroid or an aminosalicylate, such as mesalamine (e.g., DELZICOL®, ROWASA®, others), balsalazide (COLAZAL®), or olsalazine (DIPENTUM®)); an antibiotic, e.g., ciprofloxacin (cipro) or metronidazole (Flagyl); and an immune system suppressor (e.g., azathioprine (AZASAN®, IMURAN®), mercaptopurine (PURINETHOL®, PURIXAN®), methotrexate (TREXALL®), tofacitinib (XELJANZ®), upadacitinib (RINVOQ®), or ozanimod (ZEPOSIA®)). Any one or more of these therapies may optionally be used in any of the methods described herein as employing a therapy appropriate for treating UC or Crohn's disease.

In some embodiments, the respective reference level for SEQ ID NOs: 1-590 is a pre-assigned level of one of SEQ ID NOs: 1-590.

In some embodiments, the respective reference level for SEQ ID NOs: 1-590 is a level in a set of samples from a reference population. In some aspects, the reference population is a population of subjects having Crohn's disease. In other aspects, the reference population is a population of healthy subjects (e.g., a population of subjects not having an IBD (e.g., not having UC and/or not having Crohn's disease) and/or a population of subjects having a healthy gut microbiome). In some embodiments, the subjects of the reference population are human subjects.

In some embodiments, the level of each of SEQ ID NOs: 1-590 that is determined or measured in a sample from the subject is a nucleic acid level, e.g., a DNA level or an RNA level. In some embodiments, the nucleic acid level is a DNA level, which may be detected, e.g., using a PCR-based method. In other embodiments, detection of a level of one or more of SEQ ID NOs: 1-590 can optionally comprise, for example, detection of RNA levels, which can be achieved by, e.g., RT-PCR, RNA-Seq, and/or methods including the use of microarrays, as are known in the art. In other embodiments, the methods can focus on the detection of protein levels, which can be carried out using standard approaches (e.g., immunoassay-based approaches).

In some embodiments, the change in a level of one or more of SEQ ID NOs: 1-590 in the sample from the subject is a decrease relative to the respective reference level for SEQ ID NOs: 1-590 (e.g., a decrease of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, e.g., a decrease of, e.g., at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold) relative to the respective reference level for SEQ ID NOs: 1-590; or a decrease of 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, or 95-100% relative to the respective reference level for SEQ ID NOs: 1-590).

In some aspects, the levels of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of the sequences for which a level is determined or measured in the sample from the subject (i.e., one or more of SEQ ID NOs: 1-590) are decreased relative to a respective reference level for the sequence, e.g., 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, or 95-100% of the sequences for which a level is determined or measured are decreased relative to a respective reference level for the sequence.

In some embodiments, the change in a level of one or more of SEQ ID NOs: 1-590 in the sample from the subject is an increase relative to the respective reference level for SEQ ID NOs: 1-590 (e.g., an increase of at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or more than 100%, e.g., an increase of, e.g., 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold, relative to the respective level for SEQ ID NOs: 1-590; or an increase of 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, 95-100%, or more than 100%, relative to the respective reference level for SEQ ID NOs: 1-590).

In some aspects, the levels of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of the sequences for which a level is determined or measured in the sample from the subject (i.e., one or more of SEQ ID NOs: 1-590) are increased relative to a respective reference level for the sequence, e.g., 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 0-85%, 85-90%, 90-95%, or 95-100% of the sequences for which a level is determined or measured are increased relative to a respective reference level for the sequence.

In some aspects, a level of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of SEQ ID NOs: 1-590 that is increased in a sample from the subject relative to a respective reference level for SEQ ID NOS: 1-590 indicates that the subject is likely to have UC.

In some aspects, a level of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of SEQ ID NOs: 1-590 that is not substantially changed in a sample from the subject relative to a respective reference level for SEQ ID NOs: 1-590 indicates that the subject is likely to have Crohn's disease.

In another aspect, the disclosure features a method of differentiating between a diagnosis of ulcerative colitis (UC) and Crohn's disease in a subject having an inflammatory bowel disease (IBD), the method comprising determining or measuring a level of one or more of SEQ ID NOs: 1-590 in a sample from the subject, wherein (a) a level of at least 50% of SEQ ID NOs: 1-590 that is changed relative to a respective reference level for SEQ ID NOs: 1-590 indicates that the subject is likely to have UC, and (b) a level of a level of at least 50% of SEQ ID NOs: 1-590 that is not substantially changed relative to a respective reference level for SEQ ID NOs: 1-590 indicates that the subject is likely to have Crohn's disease.

In some aspects, a level of at least 50% of SEQ ID NOs: 1-590 is changed relative to a respective reference level for SEQ ID NOs: 1-590 in the sample from the subject and the method further comprises administering an anti-UC therapy to a subject.

In some aspects, a level of at least 50% of SEQ ID NOs: 1-590 is not substantially changed relative to a respective reference level for SEQ ID NOs: 1-590 in the sample from the subject and the method further comprises administering an anti-Crohn's disease therapy to a subject.

Determination of whether a difference detected is significant can be carried out using standard methods, as well as statistical analysis. In some embodiments, a difference detected is a change of at least 5%, 10%, 20%, 30%, 40%, 50%, 75%, 100%, or more, e.g., at least 2.5-fold, 3-fold, 4-fold, 5-fold, 6-fold, 7-fold, 8-fold, 9-fold, 10-fold, 15-fold, 20-fold, 25-fold, 50-fold, 75-fold, 100-fold, or more than 100-fold, relative to a reference level.

### II. ARTICLES OF MANUFACTURE AND KITS

In another aspect of the invention, an article of manufacture or kit containing materials useful for the diagnosis, prognostic assessment, and/or treatment of individuals is provided.

A kit for differentiating between a diagnosis of ulcerative colitis (UC) and Crohn's disease in a subject having an inflammatory bowel disease (IBD), the kit comprising: (a) polypeptides or polynucleotides capable of determining the level of one or more of SEQ ID NOS: 1-590 in a sample from the subject; and optionally (b) instructions for use of the polypeptides or polynucleotides to determine the level of one or more of SEQ ID NOs: 1-590 in the sample from the subject, wherein (a) a level of one or more of SEQ ID NOs: 1-590 that is changed relative to a respective reference level for SEQ ID NOs: 1-590 indicates that the subject is likely to have UC, and (b) a level of one or more of SEQ ID NOs: 1-590 that is not substantially changed relative to a respective reference level for SEQ ID NOs: 1-590 indicates that the subject is likely to have Crohn's disease.

In some aspects, the reagents for determining a level of one or more of SEQ ID NOs: 1-590 in a sample from the subject comprise one or more polynucleotides (e.g., PCR primers) that hybridize to a complement of a locus of one or more of SEQ ID NOs: 1-590 under stringent conditions and may be used to amplify all or a portion of any one or more of SEQ ID NOs: 1-590, as described herein. In some aspects, the instructions indicate that the one or more oligonucleotides (e.g., PCR primers) may be used to evaluate the presence and/or level of one or more of SEQ ID NOs: 1-590 in a sample from the subject and provide instructions for using the polynucleotide(s) for evaluating the presence and/or level of one or more of SEQ ID NOs: 1-590 in the sample. In some embodiments, reagents are included for determining a level of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or more than 99% of the sequences of SEQ ID NOs: 1-590.

For polynucleotide-based articles of manufacture or kits, the article of manufacture or kit may include, for example: (1) an oligonucleotide, e.g., a detectably labeled oligonucleotide, which hybridizes to a nucleic acid sequence encoding a protein, (2) a pair of primers useful for amplifying a nucleic acid molecule, or (3) a microarray comprising multiple oligonucleotide probes. For protein-based articles of manufacture or kits, the article of manufacture or kit may include, for example, one or more antibody-based reagents. The article of manufacture or kit can also include, e.g., a buffering agent, a preservative, or a protein-stabilizing agent. The article of manufacture or kit can further include components necessary for detecting the detectable label (e.g., an enzyme or a substrate). The article of manufacture or kit can further include components necessary for analyzing the sequence of a sample (e.g., a restriction enzyme or a buffer). The article of manufacture or kit can also contain a control sample or a series of control samples that can be assayed and compared to the test sample (e.g., a reference sample, as described herein). Each component of the article of manufacture or kit can be enclosed within an individual container and all of the various containers can be within a single package, along with instructions for interpreting the results of the assays performed using the kit.

The following examples are meant to illustrate the invention. They are not meant to limit the invention in any way.

### III. EXAMPLES

### Example 1. Identification of Markers

### A. Assembly and Annotation of Metagenomic Markers

Paired end reads from healthy Human Microbiome Project 1 (HMP1; Human Microbiome Project Consortium, Nature, 486 (7402): 207-214, 2012) individuals were downloaded from the National Center for Bioinformatics (NCBI) Short Read Archive (SRA) and assembled using metaSPAdes (cab.spbu.ru/software/spades/). Metagenomic markers were annotated using antiSMASH4.0 (docs.antismash.secondarymetabolites.org/) with the following non-default parameters: -c 3--smcogs --disable-embl. Annotated metagenomic markers were clustered using all vs. all diamond (https://github.com/bbuchfink/diamond) blastx. Blastx results were filtered using a python script requiring (a) E-value <1×10⁻⁵, (b) 90% coverage of length of coding sequence, and (c)>50% of coding sequences in a metagenomic marker present. Metagenomic markers were then grouped using markov clustering, resulting in a dereplicated library of 8,211 representative metagenomic markers identified in healthy human gut metagenomes.

### B. EMP Selection

A subset of metagenomic markers prevalent in healthy cohorts, referred to as essential microbial products (EMPs), were identified by clustering metagenomic markers presence/absence across 592 healthy patients from various geographic, genetic, and lifestyle backgrounds. Clusters with a mean prevalence >0.7 and z-score >10 within cohort were selected. To take sample imbalance into account, a proportion test was performed to assess stability across cohorts. This resulted in 1321 EMPs. For diagnostic analyses, metagenomic markers were subsetted from the full dataset, resulting in 1171 EMPs. Metagenomic markers were further subsetted, resulting in 590 EMPs.

### C. Metagenomic Data

Raw metagenomic reads from Ananthakrishnan et al., Cell Host Microbe, 21 (5): 603-610.e3, 2018 were downloaded from the NCBI Sequence Read Archive (SRA). There were 83 baseline (week 0, pretreatment) samples (43 Crohn's disease and 40 ulcerative colitis), 81 of which had baseline metagenomes available. The Ananthakrishnan et al. study presents the results of an analysis of disease activity and stool metagenomes in patients having Crohn's disease (CD) or ulcerative colitis (UC) who were treated with the anti-integrin therapy vedolizumab.

### D. Quantification of Metagenomic Markers Across Cohorts

To quantify metagenomic marker abundance across metagenomes, raw forward reads were mapped to the library of 8,211 metagenomic markers using diamond as described above. Normalized abundance of metagenomic markers was calculated such that abundance equals number of reads mapping divided by cumulative length of coding sequences in kilobase pairs divided by number of raw reads mapped.

### E. Data Preprocessing

EMP abundance data were transformed using the rank-based inverse normal transformation (Blom), and centered and scaled.

### F. Modeling

Lasso models of the form diagnosis ˜EMPs were fit using leave-one-out cross-validation with the glmnet package in R. Area under the receiver operating characteristic (ROC) curve was calculated using the pROC package in R, and the p-value of the ROC curve was computed using the Mann-Whitney method as previously described. For purposes of heatmap visualization, univariate models were fit for each EMP of the form diagnosis ˜EMP. EMPs were selected for inclusion in the heatmap on the basis of p-value of their coefficient <0.05 after Benjamini-Hochberg correction.

### G. Visualization

Heatmaps were constructed with features selected from univariate models using pheatmap with binarized data and clustering of columns and rows according to Ward's distance.

### H. Conclusions

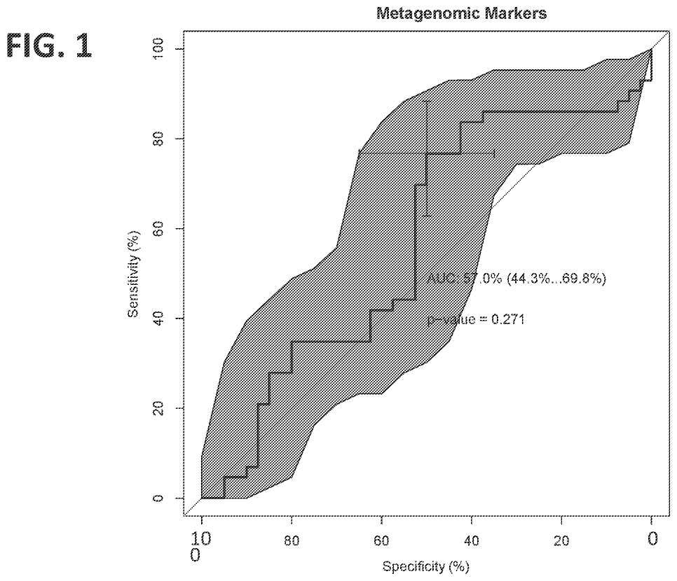

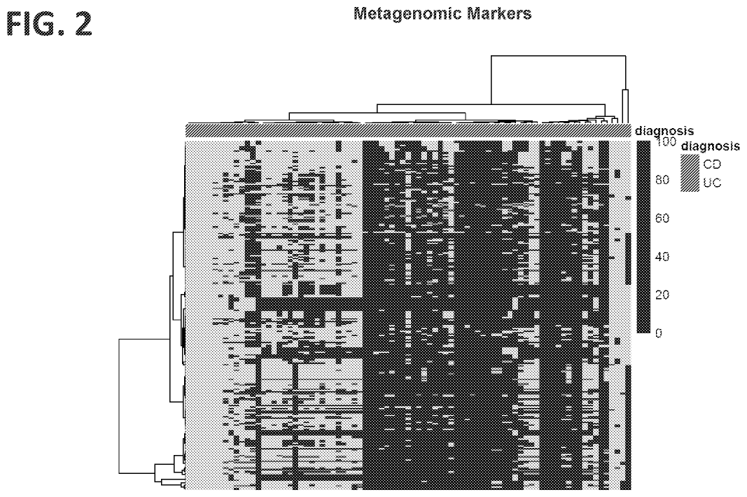

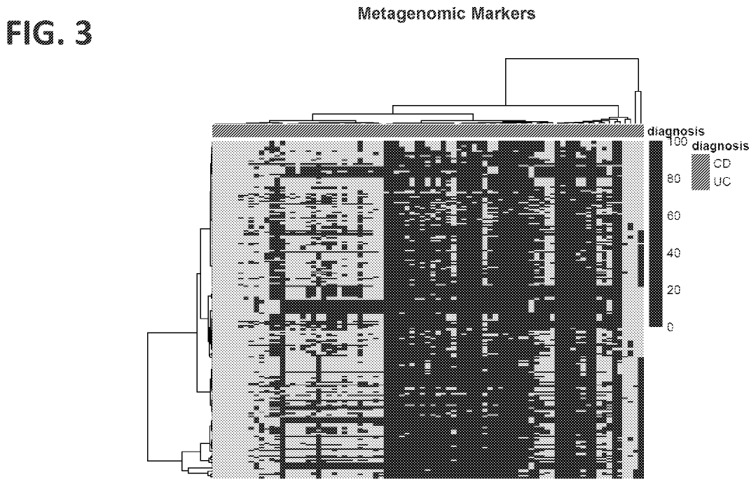

A set of 590 metagenomic markers (SEQ ID NOs: 1-590) were found to be significant predictors in a model discriminating between patients having ulcerative colitis and patients having Crohn's disease (FIGS. 1-3).

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method of differentiating between a diagnosis of ulcerative colitis (UC) and Crohn's disease in a subject having an inflammatory bowel disease (IBD), the method comprising determining a level of one or more of SEQ ID NOs: 1-590 in a sample from the subject, wherein (a) a level of one or more of SEQ ID NOs: 1-590 that is changed relative to a respective reference level for SEQ ID NOs: 1-590 indicates that the subject is likely to have UC, and (b) a level of one or more of SEQ ID NOs: 1-590 that is not substantially changed relative to a respective reference level for SEQ ID NOs: 1-590 indicates that the subject is likely to have Crohn's disease.

2. A method for determining whether a subject having an IBD has UC or Crohn's disease, comprising the steps of:
(a) measuring the nucleic acid level of one or more of SEQ ID NOs: 1-590 in a sample collected from the subject using amplification and one or more pairs of primers specific for each of the one or more of SEQ ID NOs: 1-590; and
(b) using the amplification results to determine whether the level of one or more of SEQ ID NOs: 1-590 is changed relative to a respective reference level for SEQ ID NOs: 1-590, wherein (i) a level of one or more of SEQ ID NOs: 1-590 that is changed relative to a respective reference level for SEQ ID NOs: 1-590 indicates that the subject is likely to have UC, and (ii) a level of one or more of SEQ ID NOs: 1-590 that is not substantially changed relative to a respective reference level for SEQ ID NOs: 1-590 indicates that the subject is likely to have Crohn's disease, thereby determining whether the subject has UC or Crohn's disease.

3. The method of claim 1 or 2, wherein the method comprises determining or measuring a level of at least two, at least three, at least four, at least five, at least six, at least seven, at least eight, at least nine, at least ten, at least fifteen, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, or at least 585 of SEQ ID NOs: 1-590 in the sample from the subject.

4. The method of claim 3, wherein the method comprises determining or measuring a level of all 590 of SEQ ID NOs: 1-590 in the sample from the subject.

5. The method of any one of claims 1-4, wherein a level of one or more of SEQ ID NOs: 1-590 is changed relative to the respective reference level for SEQ ID NO: 1-590 in the sample from the subject and the method further comprises administering a therapy appropriate for treating UC to the subject.

6. The method of any one of claims 1-4, wherein a level of one or more of SEQ ID NOs: 1-590 is not substantially changed relative to the respective reference level for SEQ ID NO: 1-590 in the sample from the subject and the method further comprises administering a therapy appropriate for treating Crohn's disease to the subject.

7. The method of claim 5 or 6, wherein the method further comprises treatment by fecal microbiota transplant (FMT).

8. The method of any one of claims 1-7, wherein the reference level is a pre-assigned level.

9. The method of any one of claims 1-8, wherein the reference level is a level in a set of samples from a reference population.

10. The method of claim 9, wherein the reference population is a population of subjects having Crohn's disease.

11. The method of any one of claims 1-10, wherein the level of each of SEQ ID NOs: 1-590 that is determined or measured in the sample from the subject is a nucleic acid level.

12. The method of claim 11, wherein the nucleic acid level is a DNA level.

13. The method of any one of claims 1-12, wherein the change is a decrease relative to the reference level.

14. The method of claim 13, wherein the levels of at least 10%, at least 20%, at least 30%, at least 40%, at least 50%, at least 60%, at least 70%, at least 80%, at least 90%, or more than 90% of the sequences for which a level is determined or measured are decreased relative to a respective reference level for the sequence.

15. The method of any one of claims 1-14, wherein the change is an increase relative to the reference level.

16. The method of claim 15, wherein the levels of at least 10%, at least 20%, at least 30%, at least 40%, at least 50%, at least 60%, at least 70%, at least 80%, at least 90%, or more than 90% of the sequences for which a level is determined or measured are increased relative to a respective reference level for the sequence.

17. The method of any one of claims 1-16, wherein the sample comprises a sample of the microbiota of the subject.

18. The method of any one of claims 1-17, wherein the sample is a fecal sample.

19. A kit for differentiating between a diagnosis of ulcerative colitis (UC) and Crohn's disease in a subject having an inflammatory bowel disease (IBD), the kit comprising:
(a) polypeptides or polynucleotides capable of determining the level of one or more of SEQ ID NOs: 1-590 in a sample from the subject; and
(b) instructions for use of the polypeptides or polynucleotides to determine the level of one or more of SEQ ID NOs: 1-590 in the sample from the subject, wherein (a) a level of one or more of SEQ ID NOs: 1-590 that is changed relative to a respective reference level for SEQ ID NOs: 1-590 indicates that the subject is likely to have UC, and (b) a level of one or more of SEQ ID NOs: 1-590 that is not substantially changed relative to a respective reference level for SEQ ID NOs: 1-590 indicates that the subject is likely to have Crohn's disease.

20. A method of differentiating between a diagnosis of UC and Crohn's disease in a subject having an IBD, the method comprising determining a level of each of SEQ ID NOs: 1-590 in a sample from the subject, wherein (a) a level of at least 50% of SEQ ID NOs: 1-590 that is changed relative to a respective reference level for SEQ ID NOs: 1-590 indicates that the subject is likely to have UC, and (b) a level of a level of at least 50% of SEQ ID NOs: 1-590 that is not substantially changed relative to a respective reference level for SEQ ID NOs: 1-590 indicates that the subject is likely to have Crohn's disease.

21. The method of claim 20, wherein a level of at least 50% of SEQ ID NOs: 1-590 is changed relative to a respective reference level for SEQ ID NOs: 1-590 in the sample from the subject and the method further comprises administering an anti-UC therapy to a subject.

22. The method of claim 20, wherein a level of at least 50% of SEQ ID NOs: 1-590 is not substantially changed relative to a respective reference level for SEQ ID NOs: 1-590 in the sample from the subject and the method further comprises administering an anti-Crohn's disease therapy to a subject.

23. A method of treating a subject having UC using a therapy appropriate for treating UC, wherein the subject was diagnosed as having UC by the method of any one of claims 1-18, 20, and 21.

24. A method of treating a subject having Crohn's disease using a therapy appropriate for treating Crohn's disease, wherein the subject was diagnosed as having Crohn's disease by the method of any one of claims 1-18, 20, and 22.

25. A method of treating a subject having UC using a therapy appropriate for treating UC, the method comprising the steps of:
(a) measuring the nucleic acid level of one or more of SEQ ID NOs: 1-590 in a sample collected from the subject using amplification and one or more pairs of primers specific for each of the one or more of SEQ ID NOs: 1-590;
(b) using the amplification results to determine whether the level of one or more of SEQ ID NOs: 1-590 is changed relative to a respective reference level for SEQ ID NOs: 1-590, wherein a level of one or more of SEQ ID NOs: 1-590 that is changed relative to a respective reference level for SEQ ID NOs: 1-590 indicates that the subject is likely to have UC; and
(c) treating a subject who has been determined to be likely to have UC with a therapy appropriate for treating UC.

26. A method of treating a subject having Crohn's disease using a therapy appropriate for treating Crohn's disease, the method comprising the steps of:
(a) measuring the nucleic acid level of one or more of SEQ ID NOs: 1-590 in a sample collected from the subject using amplification and one or more pairs of primers specific for each of the one or more of SEQ ID NOs: 1-590;
(b) using the amplification results to determine whether the level of one or more of SEQ ID NOs: 1-590 is changed relative to a respective reference level for SEQ ID NOs: 1-590, wherein a level of one or more of SEQ ID NOs: 1-590 that is not substantially changed relative to a respective reference level for SEQ ID NOs: 1-590 indicates that the subject is likely to have Crohn's disease; and
(c) treating a subject who has been determined to be likely to have Crohn's disease with a therapy appropriate for treating Crohn's disease.

27. The method of claim 25 or 26, wherein the nucleic acid level of the one or more of SEQ ID NOs: 1-590 in the sample collected from the subject are measured using amplification and one or more pairs of primers specific for each of the one or more of SEQ ID NOs: 1-590, and the amplification results are used to determine whether the level of one or more of SEQ ID NOs: 1-590 is changed relative to a respective reference level for SEQ ID NOs: 1-590.

28. The method of any one of claims 25-27, wherein the method comprises measuring the nucleic acid level of at least two, at least three, at least four, at least five, at least six, at least seven, at least eight, at least nine, at least ten, at least fifteen, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, at least 300, at least 310, at least 320, at least 330, at least 340, at least 350, at least 360, at least 370, at least 380, at least 390, at least 400, at least 410, at least 420, at least 430, at least 440, at least 450, at least 460, at least 470, at least 480, at least 490, at least 500, at least 510, at least 520, at least 530, at least 540, at least 550, at least 560, at least 570, at least 580, or at least 585 of SEQ ID NOs: 1-590 in the sample from the subject.

29. The method of claim 28, wherein the method comprises measuring the nucleic acid level of all 590 of SEQ ID NOs: 1-590 in the sample from the subject.

30. The method of any one of claims 25-29, wherein the reference level is a pre-assigned level.

31. The method of any one of claims 25-30, wherein the reference level is a level in a set of samples from a reference population.

32. The method of claim 31, wherein the reference population is a population of healthy subjects.

33. The method of any one of claims 25-32, wherein the nucleic acid level is a DNA level.

34. The method of any one of claims 25-33, wherein the change is a decrease relative to the reference level.

35. The method of claim 34, wherein the levels of at least 10%, at least 20%, at least 30%, at least 40%, at least 50%, at least 60%, at least 70%, at least 80%, at least 90%, or more than 90% of the sequences for which a level is measured are decreased relative to a respective reference level for the sequence.

36. The method of any one of claims 25-33, wherein the change is an increase relative to the reference level.

37. The method of claim 36, wherein the levels of at least 10%, at least 20%, at least 30%, at least 40%, at least 50%, at least 60%, at least 70%, at least 80%, at least 90%, or more than 90% of the sequences for which a level is measured are increased relative to a respective reference level for the sequence.

38. The method of any one of claims 25-37, wherein the sample comprises a sample of the microbiota of the subject.

39. The method of any one of claims 25-38, wherein the sample is a fecal sample.

40. A method of treating a subject having UC, the method comprising the steps of:
(a) measuring the nucleic acid level of each of SEQ ID NOs: 1-590 in a sample collected from the subject, wherein a level of at least 50% of SEQ ID NOs: 1-590 that is changed relative to a respective reference level for SEQ ID NOs: 1-590 indicates that the subject is likely to have UC, thereby determining an increased risk of UC for the subject; and
(b) treating a subject who has been determined to have an increased risk of UC with a therapy appropriate for treating UC.

41. A method of treating a subject having Crohn's disease, the method comprising the steps of:
(a) measuring the nucleic acid level of each of SEQ ID NOs: 1-590 in a sample collected from the subject, wherein a level of at least 50% of SEQ ID NOs: 1-590 that is not substantially changed relative to a respective reference level for SEQ ID NOs: 1-590 indicates that the subject is likely to have Crohn's disease, thereby determining an increased risk of Crohn's disease for the subject; and
(b) treating a subject who has been determined to have an increased risk of Crohn's disease with a therapy appropriate for treating Crohn's disease.

42. The method of claim 41 or 42, wherein the nucleic acid levels of each of SEQ ID NOs: 1-590 in the sample collected from the subject are measured using amplification and one or more pairs of primers specific for each of SEQ ID NOs: 1-590, and the amplification results are used to determine whether the level of each of SEQ ID NOs: 1-590 is changed relative to a respective reference level for SEQ ID NOs: 1-590.

43. The method of any one of claims 5, 6, 23 to 26, 40, and 41, wherein the therapy appropriate for treating UC or Crohn's disease comprises an anti-inflammatory drug, a biologic therapy, an anti-inflammatory, an immune system suppressor, a steroid, an analgesic, an antibiotic, surgery, and/or fecal microbiota transplant (FMT).

44. The method of claim 43, wherein the anti-inflammatory is a nonsteroidal anti-inflammatory drug, a corticosteroid, or an aminosalicylate (e.g., an oral 5-aminosalicylate, such as sulfasalazine or mesalamine); the biologic therapy is vedolizumab (ENTYVIO®), infliximab (REMICADE®), adalimumab (HUMIRA®), golimumab (SIMPONI®), certolizumab (CIMZIA®), risankizumab (SKYRIZI®), ustekinumab (STELARA®), natalizumab, pegol, or ustekinumab; the antibiotic is ciprofloxacin (cipro) or metronidazole (Flagyl); or the immune system suppressor is azathioprine (AZASAN®, IMURAN®), mercaptopurine (PURINETHOL®, PURIXAN®), methotrexate (TREXALL®), tofacitinib (XELJANZ®), upadacitinib (RINVOQ®), or ozanimod (ZEPOSIA®).
